(19) United States
(12) Patent Application Publication
(10) Pub. No.: US 2015/0182732 A1
(43) Pub. Date: Jul. 2, 2015

(54) Drug Eluting Balloon With Preferred Drug Orientation To Improve Drug Transfer Efficiency

(71) Applicant: Boston Scientific Scimed, Inc., Maple Grove, MN (US)

(72) Inventors: Hongxia Zeng, Maple Grove, MN (US); Yen-Lane Chen, New Brighton, MN (US); Steven L. Kangas, Woodbury, MN (US)

(73) Assignee: Boston Scientific Scimed, Inc., Maple Grove, MN (US)

(21) Appl. No.: 14/583,941

(22) Filed: Dec. 29, 2014

Related U.S. Application Data
(60) Provisional application No. 61/923,068, filed on Jan. 2, 2014.

(51) Int. Cl. A61M 25/10

(52) U.S. Cl. CPC A61M 25/1029; A61M 2025/1031

(57) ABSTRACT

A catheter comprises a medical balloon having a drug coating. The drug coating comprises drug crystals on a surface of the balloon. The majority of the drug crystals on the surface of the balloon are oriented drug crystals which extend within 5° of a predetermined, non-zero common angle relative to the surface of the balloon from which the crystals extend.

### CROSS-REFERENCE TO RELATED APPLICATIONS

This Application claims the benefit of and priority to U.S. Provisional Application No. 61/923,068, filed Jan. 2, 2014, the entire contents of which are herein incorporated by reference.

### STATEMENT REGARDING FEDERALLY SPONSORED RESEARCH

Not Applicable.

### BACKGROUND OF THE INVENTION

Drug coated balloons, may include crystalline drugs or other types of drug particles disposed on the balloon. The drug crystals or drug particles are typically randomly orientated on the balloon surface. Some of the coating, however, may be lost during tracking of the balloon and some of the coating may be lost during deployment. This may result in a relatively small percentage, between 1% and 10%, of the drug being deposited on an artery or other vessel. Thus, there may be low drug transfer efficiency.

It is believed that the solid particulates that are deposited on the wall of the artery or other vessel have three potential fates. Some are likely flushed from the artery wall into the blood stream. Those that remain in contact with the artery wall will slowly dissolve. Some fraction will dissolve into the blood stream. Some fraction will be taken up by the vessel. Very small particles which are less than one micron in size can be taken up directly into the arterial tissue. Some of the drug that diffuses into the vessel wall is believed to bind to and stabilize the cell microtubules, thereby affecting the restenotic cascade after injury of the artery.

It would be beneficial to have drug coated medical devices with novel coatings.

### BRIEF SUMMARY OF VARIOUS EMBODIMENTS OF THE INVENTION

In at least one embodiment, a catheter comprises a medical balloon having a drug coating. The drug coating comprises drug crystals. A majority of the drug crystals are oriented drug crystals which extend within 45°, and, in some embodiments, within 20°, and, in some embodiments, within 10° and, desirably, within 5°, of a predetermined common angle relative to a surface of the balloon. Desirably, the oriented drug crystals extend within 45°, and, more desirably, within 20°, and, even more desirably, with 10°, and, more desirably yet, within 5° perpendicular to the surface of the balloon. More desirably, 90 percent or more of the drug crystals are oriented drug crystals which extend within 45°, and, more desirably, within 20°, and, even more desirably, with 10°, and, more desirably yet, within 5° of a predetermined angle relative to a surface of the balloon. More desirably, 90 percent or more of the drug crystals are oriented drug crystals which extend within 45°, and, more desirably, within 20°, and, even more desirably, with 10°, and, more desirably yet, within 5° of perpendicular to a surface of the balloon.

In at least one embodiment, a method of preparing a catheter comprises the step of providing a catheter comprising a medical balloon. The balloon has topological features which define one or more regions of the balloon having a depth of at least 50 microns and up to 500 microns. A drug is disposed within the topological features and oriented drug crystals are formed within the topological features. A majority of the drug crystals are oriented drug crystals which extend within 5° of a predetermined common angle relative to a surface of the balloon. Desirably, the oriented drug crystals extend within 5° perpendicular to the surface of the balloon.

The balloon may comprise a polymeric material and the topological features may be provided in the polymeric material.

The balloon may comprise a template formed from aggregated surfactants arranged in lamellar structures. The topological features are provided by the lamellar structures. The template is typically removed subsequent to forming the drug to form oriented drug crystals.

In at least one embodiment, a catheter comprises a medical balloon having a drug coating. The drug coating comprises oriented drug crystals wherein the orientation of the drug crystals is not random. Typically, the drug crystals will be oriented within 5° of perpendicular to the surface of the balloon. The balloon may comprise a template formed from aggregated surfactants arranged in lamellar structures, the drug crystals disposed in the template.

These and other aspects, embodiments and advantages of the present disclosure will become immediately apparent to those of ordinary skill in the art upon review of the Detailed Description and Claims to follow.

### BRIEF DESCRIPTION OF THE SEVERAL VIEWS OF THE DRAWING(S)

FIG. 1 is a perspective view of a balloon catheter that includes an inflated, coated balloon.

FIG. 2 shows a magnified view of a portion of the balloon of FIG. 1.

FIG. 3 illustrates a single drug crystal which extends from a balloon surface at a perpendicular angle to the surface.

FIG. 4a illustrates a single drug crystal which extends from a balloon surface at an oblique angle to the surface.

FIG. 4b illustrates, schematically, a crystal which extends within 0 degrees of an angle α relative to a surface of a balloon.

FIG. 5 is a schematic diagram showing a method of coating a medical balloon.

FIG. 6 is a schematic diagram showing a method of coating a medical balloon.

### DETAILED DESCRIPTION OF THE INVENTION

While embodiments of the present disclosure may take many forms, there are described in detail herein specific embodiments of the present disclosure. This description is an exemplification of the principles of the present disclosure and is not intended to limit the disclosure to the particular embodiments illustrated.

For the purposes of this disclosure, a crystal which extends perpendicular to a surface is characterized as having a longitudinal axis which is normal to the surface in the region of the surface from which the crystal extends. This means that the longitudinal axis of the crystal is perpendicular to any tangent which lies along the surface at the location from which the crystal extends. Also, the term ‘within n° ’ of an angle means within ±n° of the angle.

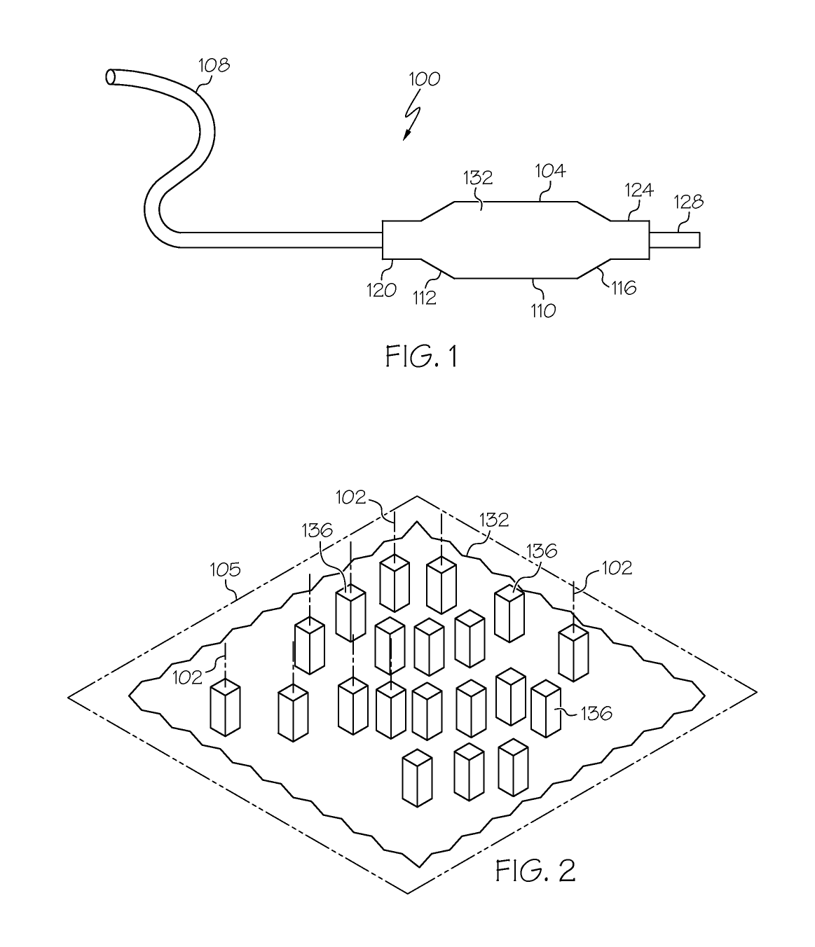

FIG. 1 depicts the distal end of a balloon catheter, shown generally at 100. Balloon catheter 100 includes a balloon, 104, shown in an inflated state. Balloon 104 extends from catheter tube 108. Balloon 104 includes body portion 110, cone portions 112 and 116 at the proximal and distal ends thereof and waist portions 120 and 124. Balloon catheter 100 terminates in a distal tip 128 and includes an inflation lumen which is in fluid communication with the balloon and may optionally include a guidewire disposed therein. Any suitable balloon catheter configuration known in the art may be used, including those disclosed in U.S. Pat. No. 6,036,697, incorporated herein in its entirety by reference. The catheter and balloon may be made of any suitable materials including the materials disclosed in U.S. Pat. No. 8,034,280 and U.S. Pat. No. 8,025,636, the contents of both of which are incorporated herein in their entirety.

Drug coated balloons may include crystalline drugs or other types of drug particles disposed on the balloon. The drug crystals or drug particles are typically randomly orientated on the balloon surface. This may result in low drug transfer efficiency.

In one or more embodiments, a catheter with a medical balloon is disclosed. The balloon comprises a coating comprising a crystalline drug. The coating may extend over the entire outer surface of the medical balloon or over less than the entire outer surface of the balloon. The coating may be provided in one or more regions of the balloon.

The coating may consist of drug crystals or may include additional components. Typically, the coating will be located on an outer surface of the balloon. An outer surface of a balloon refers to that portion of a balloon which is exposed to bodily fluids and tissues.

A majority of the drug crystals on the surface of the balloon are oriented drug crystals which extend within 45°, more desirably, within 20°, even more desirably, within 10°, even more desirably 5°, and even more desirably, 1° of an axis which extends at a predetermined, non-zero common angle relative to the surface of the balloon from which the crystals extend.

In one or more embodiments, 75 percent or more of the drug crystals on the surface of the balloon are oriented drug crystals which extend within 45°, more desirably, within 20°, even more desirably, within 10°, even more desirably 5°, and even more desirably, 1° of a an axis which extends at a predetermined, non-zero common angle relative to the surface of the balloon from which the crystals extend.

In one or more embodiments, 90 percent or more of the drug crystals on the surface of the balloon are oriented drug crystals which within extend 45°, more desirably, within 20°, even more desirably, within 10°, even more desirably 5°, and even more desirably, 1° of an axis which extends at a predetermined, non-zero common angle relative to the surface of the balloon from which the crystals extend.

In one or more embodiments, 95 percent or more of the drug crystals on the surface of the balloon are oriented drug crystals which extend within 45°, more desirably, within 20°, even more desirably, within 10°, even more desirably 5°, and even more desirably, 1° of an axis which extends at a predetermined, non-zero common angle relative to the surface of the balloon from which the crystals extend.

In one or more embodiments, 99 percent or more of the drug crystals on the surface of the balloon are oriented drug crystals which extend within 45°, more desirably, within 20°, even more desirably, within 10°, even more desirably 5°, and even more desirably, 1° of an axis which extends at a predetermined, non-zero common angle relative to the surface of the balloon from which the crystals extend.

Desirably, in all of the embodiments disclosed herein, the oriented crystals are all substantially parallel with one another. Also desirably, in all of the embodiments disclosed herein, the long axes of all of the oriented crystals extend at an angle within 5 degrees of one another relative the surface of the medical device.

The axis which extends at predetermined, non-zero common angle relative to the surface of the balloon from which the oriented crystals extend may extend at any desired angle. Typically, however, the predetermined angle will be any angle from 45° to 90°. Desirably, the predetermined angle will be any angle from 60° to 90°. Even more desirably, the predetermined angle will be 90°.

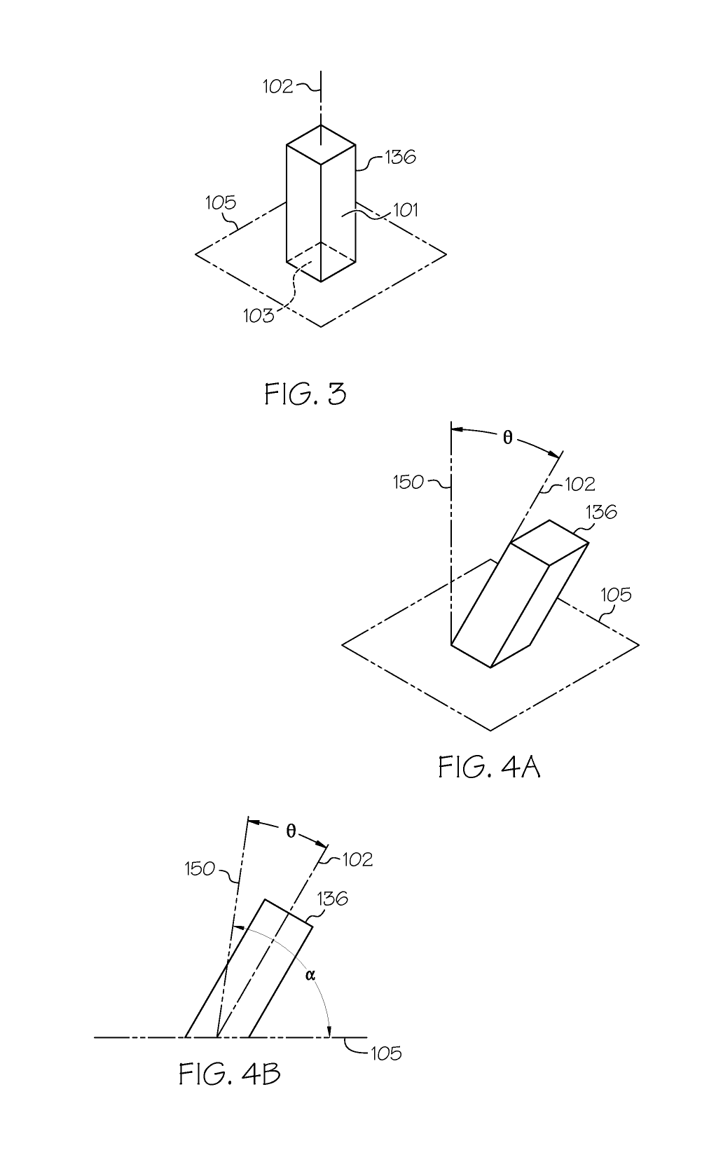

FIG. 2 shows a magnified view of a portion 132 of balloon 104 depicting drug crystals 136 which extend in a direction perpendicular to the surface 105 of balloon 104. FIG. 3 illustrates a single drug crystal 136 which extends from a balloon surface 105 at a perpendicular angle to the surface. FIG. 4a illustrates a single drug crystal 136 which extends from a balloon surface 105 at an oblique angle Θ relative to an axis which extends in a direction perpendicular to the surface. Thus, crystal 136 extends within Θ degrees of an axis which extends in a direction perpendicular to the surface. FIG. 4b illustrates, schematically, a crystal which extends within Θ degrees of an axis 150 which extends at an angle α relative to surface 105 of a balloon.

Desirably, the drug crystals will be from 5-500 microns. This means that the longest side of the crystal is from 5-500 microns. More desirably, the longest side of the drug crystals will be from 10-100 microns. Optionally, the crystals may be smaller or larger than the above-recited range.

The orientation of the drug crystals on the balloon surface, or on the surface any other suitable medical device, can be controlled by templates formed by surfactant aggregation. In one or more embodiments, a surfactant-based template is formed by using a surfactant disposed in water with a surfactant concentration above the critical micelle concentration. The surfactant can be ionic such as cetyltrimethyl ammonium template (CTAB), and sodium dodecyl sulfate, or non-ionic, such as polyoxyethylene glycol alkyl ethers (Brij surfactants), polyethylene glycol surfactants (PEG), and Alkylphenol hydroxypolyethylene (Triton surfactants), or zwitterionic such as 1,2-dioleoylphosphatidylcholine (DOPC), 1-palmitoyl-2-oleoyl-sn-glycero-3-phosphocholine (POPC), and dipalmitoylphosphatidylcholine (DPPC). A lamellar structure can be formed from surfactant aggregations.

The resulting template may then be dip coated onto a balloon surface or onto the surface of any other suitable device including a stent, graft, graft or implantable valve. The template may be seeded with nuclei containing newly forming crystals of the drug. The template constrains the drug crystals to grow only in one dimension which results in drug bundles.

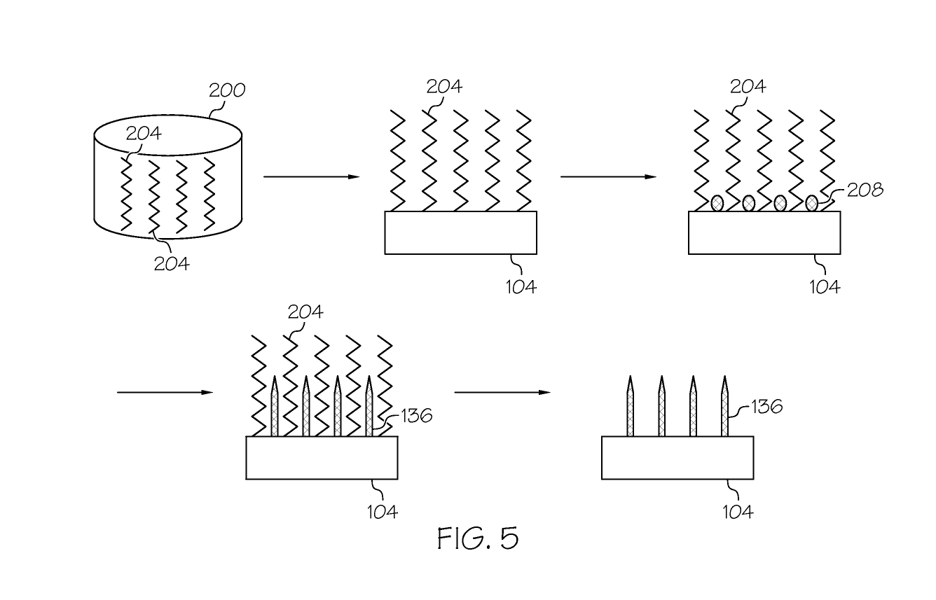

This method is depicted schematically in FIG. 5. A vessel 200 contains a surfactant with a concentration above the critical micelle concentration. Aggregated surfactants, shown at 204, form a lamellar structure. A balloon 104 is then dip coated in the surfactant and the aggregated surfactants are disposed on the surface of balloon 104 forming a template thereon. Nuclei 208 are then introduced onto the surface of the balloon and crystals 136 are grown. Aggregated surfactants 204 forming the template are then washed off balloon 104 with a suitable solvent, leaving oriented drug crystals 136 extending perpendicularly from the surface of balloon 104.

In order to ensure that the crystals retain the desired orientation, it is desirable for there to be a sufficient density of crystals on the surface of the balloon or other medical device in the region of the crystals. The minimum density of crystals on the surface of the balloon, or other medical device, in the region with the crystals, i.e. crystals per unit area of the surface of the balloon or medical device, will depend, at least in part, on the size of the crystals and the size of the region having the crystals. As the density of crystals (ie number of crystals per unit area) in the region of the surface with the crystals increases, the crystals will be more likely to retain their orientation if the surface is disturbed, such as by rinsing. Desirably, the crystal density will be in the range of 1 μg/mm² to 5 μg/mm². Also desirably, there will be from 5-25 crystals/100 μm². More desirably, there will be from 10-15 crystals/100 μm². Typically, the crystals will have a diameter or other width dimension on the order of several microns.

Drug nuclei can be provided by over-saturated drug solution in various solvents including water, ethyl acetate/acetone/n-hexane, ethyl acetate/heptane, tetrahydrofuran (THF)/Heptane, isopropyl alcohol (IPA), para-xylene and cyclohexanone, acetone, acetone/water, IPA/THF, acetonitrile, 2-butanone, isopropyl ether (IPE), diethyl ether (DEE), methyl isobutyl ketone (MIBK), monofluorobenzene (MFB), a,a,atrifluorotoluene (TFT), nitromethane (NM), and ethyl trifluroacetate (ETFA).

The growth of drug crystalline bundles can be controlled by slow evaporation, nuclei density, temperature, and vapor pressure. When growth of drug crystalline is finished, the balloon or other suitable medical device, including a stent, graft or implantable valve, can be rinsed by water to dissolve the surfactant and only drug crystals with the desired orientation are left.

Crystalline drug may also be created by first depositing amorphous nanoparticles of drug into the template using a solvent, for example, ethanol, ethylacetate/heptane, acetone, IPA, MIBK, DEE, 2,2,2,-trifluroethanol (TFE), TFT, MFB, chloromethane (CH₃Cl), or trichloroethylene (TCE).

In at least one embodiment, a template controls the orientation of drug crystals as the crystals grow. Typically, the template will provide only one dimension in which the drug crystals may grow. This alignment allows the drug to exist on the balloon or other medical device, including a stent, graft or implantable valve, in the form of parallel crystalline strands of crystals on the surface of the balloon or other medical device.

Once the crystals have grown to the desired size, the template may be washed off with a suitable solvent. For example, for drugs which exhibit minimum solublity in water, water may be used to wash off the template from the balloon or other medical device, including a stent, graft or implantable valve.

In one or more embodiments, saturated drug solution may be added into a self-assembled surfactant solution. The surfactant and drug solution may then be dip coated onto a balloon or other medical device, including a stent, graft or implantable valve. The coating is then allowed to slowly dry to induce crystallization of the drug. The resulting drug infused template constrains crystals of the drug to grow only in one dimension, resulting in drug bundles. When growth of the drug crystals is finished, the balloon or other medical device can be rinsed with a suitable solvent such as water to dissolve the surfactant, leaving only the crystalline drug with the desired orientation on the surface of the balloon or other medical device.

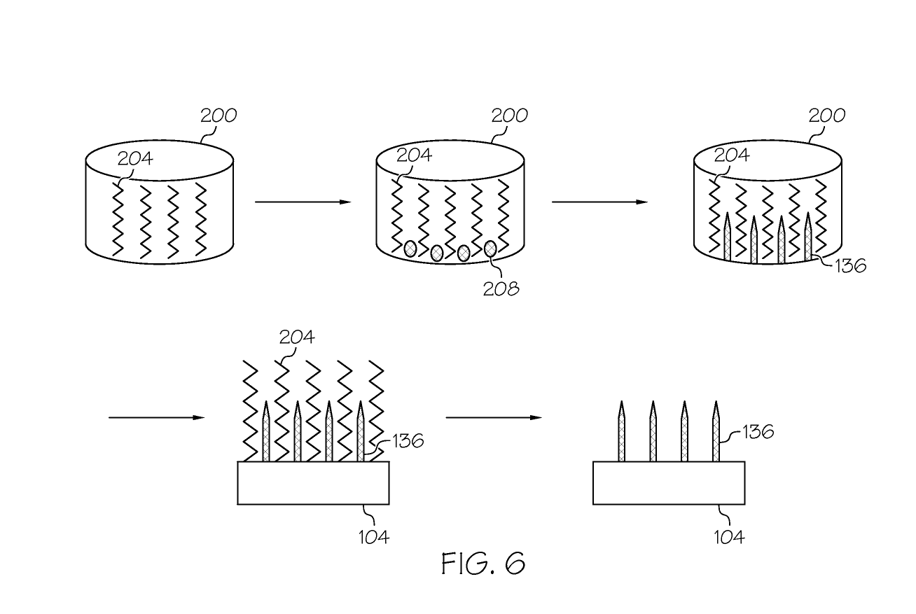

A modified form of this method is depicted schematically in FIG. 6. Vessel 200 contains a surfactant with a concentration above the critical micelle concentration. Aggregated surfactants, shown at 204, form a lamellar structure. Nuclei 208 are then introduced between strands of aggregated surfactants 204 and a saturated drug solution is added to the to the surfactant solution. Crystals 136 are, optionally, grown within the template formed of the aggregated surfactants.

A balloon 104 is then dip coated in the surfactant and the aggregated surfactants, optionally containing drug crystals, are disposed on the surface of balloon 104 forming a template thereon. If crystals were not previous grown in the template, crystals are then grown. If crystals were previously grown in the template, they are, optionally, allowed to continue to grow to a desired size. Aggregated surfactants 204 forming the template are then washed off balloon 104 with a suitable solvent, leaving oriented drug crystals 136 extending perpendicularly from the surface of balloon 104.

When the balloon is being dip coated, the balloon can be in the as-formed state, in a partially inflated state or in a totally inflated state. Where the balloon is in a partially inflated stated, it will typically be pressurized to 1 to 2 atmospheres pressure, although the balloon may be pressurized to higher or lower pressures. The balloon may be in an unfolded configuration or a creased, partially folded or folded configuration.

In yet another embodiment, a microstructured polymer is used as a template to control morphology of the crystalline drug. Polymer films, for example, polyvinylpyrrolidone (PVP), polystyrene (PS), and poly(butyl methacrylate) (PBMA), with an array of topological features such as porous channels, grids or lines (desirably micron-sized) can be provided as a substrate on the surface of a balloon or other medical device including stent, graft or implantable valve. Crystalline drug seeds may be embedded within the topological features. Subsequent crystallization of the drug within the topological features on the surface of the balloon or other medical device could generate drug bundles. Details about the use of crystalline drug seeds may be found in US Publication 20130053947, incorporated herein in its entirety by reference.

In yet another embodiment, a template can be provided directly on the surface of the balloon or other medical device such as a stent, graft or implantable valve. For example, a pattern such as a disk, grid, or square may be printed on the surface of a balloon or other medical device. The depth of these patterns desirably ranges from 5 microns up to 500 microns. The patterns may be used as a deposit site to load crystalline seeds. Further growth of crystalline drug is controlled and limited within the template which has been provided in the material of the balloon or other medical device.

The patterns may be printed using polymers or other suitable materials including inorganic materials such as salts and organic materials such as sugars. Desirably, the polymers or other suitable materials will be soluble in a solvent in which the drug is insoluble or substantially insoluble so that the pattern may, optionally, be removed from the balloon or other medical device once crystal growth has been completed. For example, where the desired drug is paclitaxel, a polymer which is soluble in water may be printed onto the surface of a balloon or other medical device. Once the paclitaxel crystals are grown, the water soluble polymer template may be washed away with water, leaving the paclitaxel crystals intact on the surface of the balloon or other medical device.

Polymers which are suitable include polyvinyl pyrolidone (PVP), polyethylene oxide (PEO). Other suitable organic materials include sugars such as sucrose. Suitable inorganic materials include salts such as sodium chloride.

Patterns may be introduced directly into the balloon material by using laser ablation.

Details about printing on medical devices may be found in U.S. Pat. No. 6,676,987 and U.S. Pat. No. 6,841,213, both of which are incorporated herein by reference in their entirety.

Additionally, details concerning providing microstructures on surfaces may be found in US patent publication 20130268063, incorporated herein in its entirety by reference.

Any suitable technique, including those disclosed in US patent publications US 20130053947, 20110015664, 20100272773, 20060088566, incorporated herein in their entirety be reference, may be used to crystallize drugs for use in any of the embodiments disclosed herein. Examples of suitable techniques include the following:

### Slurry Crystallization

A drug powder may be suspended in polar solvent. Anti-solvents in the form of less polar solvents are added and the sample agitated and dried.

Solvent systems which may be used to convert everolimus into crystalline form include:

ethyl acetate/acetone/n-hexane,

ethyl acetate/heptane,

tetrahydrofuran (THF)/Heptane,

isopropyl alcohol,

para-xylene,

cyclohexanone,

ethanol/glycerol, and

IPA/glycerol.

The solvent/anti-solvent/drug solution may be applied onto a polymer substrate on the surface of a balloon or other medical device including a stent, graft or implantable valve and grown into drug bundles having crystals of the drug.

Nucleation from Solvent Mixture

A drug may be dissolvent in a solvent and then crystallized via a slow drying process.

Everolimus, for example, may be dissolved in solvent and crystallized with a slow drying process. Suitable solvent systems include isopropyl alcohol, acetone, acetone/water, isopropyl alcohol/tetrahydrofuran, acetonitrile, 2-butanone, isopropyl ether, diethyl ether, methyl isobutyl ketone, monofluorobenzene, a,a,a-trifluorotoluene, nitromethane, ethyl trifluroacetate, ethanol/glycerol, and IPA/glycerol.

The everolimus crystalline seeds can be placed into the template for further growth.

### Vapor Stress

A drug solution, optionally over-saturated, may be placed into a polymer and exposed to a vapor laden environment.

For example, an amorphous everolimus solution, optionally over-saturated, is placed into a polymer pattern and exposed to a vapor laden environment to facilitate growth of everolimus crystals. Vapor systems include ethylacetate/heptane, acetone, isopropyl alcohol, methyl isobutyl ketone, diethyl ether, 2,2,2,-trifluroethanol, a,a,atrifluorotoluene, monofluorobenzene, chloromethane (CH₃Cl), trichloroethylene.

In general, the growth of crystalline drug can be controlled by solvent evaporation, vapor annealing, density of nuclei or any other suitable technique.

Any suitable drug which forms crystals may be used with any of the medical devices disclosed herein, including balloons, stents and valves. Examples of drugs which may used include paclitaxel, and the olimus rugs including sirolimus (rapamycin), everolimus, zotarolimus, Biolimus A9 (Biosensors International, Singapore), AP23572 (Ariad Pharmaceuticals), tacrolimus, pimecrolimus, deferolimus, temsirolimus, and derivates or analogs of any of these above-mentioned drugs. Where paclitaxel is used, desirably the paclitaxel will comprise the crystalline dihydrate form of paclitaxel. In some embodiments, the paclitaxel will comprise the crystalline dihydrate form of paclitaxel as well as the anhydrous crystalline form of paclitaxel. In other embodiments, the paclitaxel will consist of the crystalline dihydrate form of paclitaxel and the anhydrous crystalline form of paclitaxel. In yet other embodiments, the paclitaxel will consist of the crystalline dihydrate form of paclitaxel.

Where paclitaxel is used, and in particular, the dihydrate crystalline form, suitable solvent systems include:

a combination of methanol and water;

a combination of acetone and water.

The ratio of water to methanol can range from 50:50 to 1:99 by volume. Similarly, the ratio of water to acetone can range from 50:50 to 1:99 by volume.

Additional details concerning paclitaxel may be found in US Patent Publications 20100272773, 20110015664, 20110008260 and 20130053947, incorporated herein in their entirety by reference.

The invention, in one or more embodiments, is directed to the inventive balloons disclosed herein as well as to balloon catheters comprising any of the inventive balloons disclosed herein.

The inventive balloon and balloon catheters and other medical devices disclosed herein may be used for treatment of coronary artery disease or peripheral artery disease or for any other suitable treatment within the body.

Although various embodiments have been discussed with respect to balloons and catheters with medical balloons, other embodiments of the invention are directed to other medical devices as well including stent, grafts, filters, and implantable valves. By way of non-limiting examples, the one or more embodiments of the invention are directed to stents which can be coated with drugs, grafts (including stent grafts) which can be coated with drugs, filters which can be coated with drugs and implantable valves which can be coated with drugs. Examples of stents are shown in U.S. Pat. No. 6,896,696, U.S. Pat. No. 6,818,014, U.S. Pat. No. 8,142,489 and US Publication 20070073384, all which are incorporated herein in their entirety by reference. Examples of valves are given in U.S. Pat. No. 8,231,670 and US Publication 20050137688, both of which are incorporated herein in their entirety by reference. Examples of filters are given in U.S. Pat. No. 7,481,823, incorporated herein in its entirety by reference. Examples of grafts are given in US 20100152833 incorporated herein in its entirety by reference.

Any of the devices discussed herein may be provided with oriented drug crystals using any of the techniques discussed above. Thus, stent, grafts, filters which deliver drugs, and implantable valves which deliver drugs may be provided with topological features and drug disposed within the topological features. As discussed above, the topological features may be printed on the surface of the device or they may be provided via a surfactant-based template, as discussed above. Details about printing on medical devices may be found in U.S. Pat. No. 6,676,987 and U.S. Pat. No. 6,841,213 both of which are incorporated herein by reference in their entirety.

Any of the inventive balloons and balloon catheters as well as other medical devices disclosed herein may have the above-mentioned oriented drug crystals disposed over the entirety of the medical device or only over a portion. For balloons and balloon catheters, the oriented drug crystals may be disposed on the entirety of the outer surface of the balloon or only over a portion. The oriented drug crystals may be disposed on the entirety of the outer surface of the body portion of the balloon or only over a portion of the body portion of the balloon. The oriented drug crystals may be disposed on the entirety of the outer surface of one or more cone portions of the balloon or only over a portion of one or more cone portions of the balloon. The oriented drug crystals may be disposed on the entirety of the outer surface of one or more waist portions of the balloon or only over a portion of one or more waist portions of the balloon.

Similarly, where the medical device is a stent, graft, or implantable valve, the oriented drug crystals may be disposed over the entirety of the device or only over a portion of the device.

The invention is also directed, in one or more embodiments, to the following numbered statements:

1. A catheter comprising a medical balloon having a drug coating, the drug coating comprising drug crystals on a surface of the balloon, a majority of the drug crystals on the surface of the balloon being oriented drug crystals which extend within 45°, more desirably, within 20°, even more desirably, within 10°, even more desirably 5°, and even more desirably, 1° of an axis which extends at a predetermined, non-zero common angle relative to the surface of the balloon from which the crystals extend.
2. The catheter of statement 1 wherein the oriented drug crystals extend within 5° of an axis which extends perpendicular to the surface of the balloon.
3. The catheter of statement 1 wherein 90 percent or more of the drug crystals on the surface of the balloon are oriented drug crystals which extend within 45°, more desirably, within 20°, even more desirably, within 10°, even more desirably 5°, and even more desirably, 1° of an axis which extends at a predetermined, non-zero common angle relative to the surface of the balloon from which the crystals extend.
4. The catheter of statement 3 wherein the oriented drug crystals extend within 5° of an axis which extends perpendicular to the surface of the balloon.
5. A method of preparing a catheter comprising the steps of:

providing a catheter comprising a medical balloon, the balloon having topological features, each of the features defining a region of the balloon having a depth of at least 5 microns and up to 500 microns;

disposing a drug within the topological features;

forming drug crystals within the topological features;

wherein a majority of the drug crystals are oriented drug crystals which extend within 45°, more desirably, within 20°, even more desirably, within 10°, even more desirably 5°, and even more desirably, 1° of a predetermined common angle relative to a surface of the balloon.

6. The method of statement 5 wherein the oriented drug crystals extend within 5° of an axis which extends perpendicular to the surface of the balloon.
7. The method of any of statements 5 or 6 wherein the balloon comprises a polymeric material and the topological features are provided in the polymeric material.
8. The method of any of statements 5 or 6 wherein the balloon comprises a template formed from aggregated surfactants arranged in lamellar structures, the topological features provided by the lamellar structures; and removing the template subsequent to forming oriented drug crystals.
9. The method of statement 8 including removing the template by applying water thereto.
10. The method of any of statements 8 or 9 wherein the surfactants are selected from the group consisting of ionic, non-ionic, and zwitterionic surfactants.
11. The method of any of statements 8, 9 or 10 wherein the surfactant is ionic.
12. The method of statement 8, 9, 10 or 11 wherein the surfactant is cetyltrimethyl ammonium template or sodium dodecyl sulfate.
13. The method of any of statements 8, 9, or 10 wherein the surfactant is non-ionic.
14. The method of statement 8, 9, 10 or 13 wherein the surfactant is selected from the group consisting of polyoxyethylene glycol alkyl ether surfactants, polyethylene glycol surfactants and alkylphenol hydroxypolyethylene surfactants.
15. The method of statement 8, 9, or 10 wherein the surfactant is zwitterionic.
16. The method of statement 8, 9, 10 or 15 wherein the surfactant is selected from the group consisting of 1,2-dioleoylphosphatidylcholine, 1-palmitoyl-2-oleoyl-sn-glycero-3-phosphocholine, and dipalmitoylphosphatidylcholine.
17. A catheter comprising a medical balloon having a drug coating, the drug coating comprising drug crystals, a majority of the drug crystals being oriented drug crystals wherein the orientation of the drug crystals is not random.
18. The catheter of statement 17 wherein the drug crystals are oriented within 15° of perpendicular to the surface of the balloon.
19. The catheter of any of statements 17 or 18 wherein the balloon comprises a template, the template formed from aggregated surfactants arranged in lamellar structures, the drug crystals disposed in the template.
20. The catheter of any of statements 17, 18 or 19 wherein at least 90 percent of the drug crystals are oriented within 45°, more desirably, within 20°, even more desirably, within 10°, even more desirably 5°, and even more desirably, 1° of an axis which extends perpendicular to the surface of the balloon.
21. A medical balloon having a drug coating, the drug coating comprising drug crystals, the majority of the drug crystals being oriented drug crystals which extend within 45°, more desirably, within 20°, even more desirably, within 10°, even more desirably 5°, and even more desirably, 1° of an axis which extends at a predetermined, non-zero common angle relative to the surface of the balloon from which the crystals extend.
22. The medical balloon of statement 21 wherein the oriented drug crystals extend within 15° of an axis which extends perpendicular to the surface of the balloon.
23. The medical balloon of statement 21 wherein 90 percent or more of the drug crystals on the surface of the balloon are oriented drug crystals which extend within 15° of an axis which extends at a predetermined, non-zero common angle relative to the surface of the balloon from which the crystals extend.
24. The medical balloon of statement 23 wherein oriented drug crystals extend within 5° of an axis which extends perpendicular to the surface of the balloon.
25. A method of preparing a balloon comprising the steps of:

providing a medical balloon, the balloon having topological features, the topological features defining one or more regions of the balloon having a depth of at least 5 microns and up to 500 microns;

disposing a drug within the topological features;

forming oriented drug crystals within the topological features;

wherein a majority of the drug crystals are oriented drug crystals which extend within 45°, more desirably, within 20°, even more desirably, within 10°, even more desirably 5°, and even more desirably, 1° of an axis which extends at a predetermined common angle relative to a surface of the balloon.

26. The method of statement 25 wherein the oriented drug crystals extend within 15° of an axis which extends perpendicular to the surface of the balloon.
27. The method of any of statements 25 or 26 wherein the balloon comprises a polymeric material and the topological features are provided in the polymeric material.
28. The method of statement 25 or 26 wherein the balloon comprises a template formed from aggregated surfactants arranged in lamellar structures, the topological features provided by the lamellar structures; and removing the template subsequent to forming oriented drug crystals.
29. The method of statement 28 including removing the template by applying water thereto.
30. The method of any of statements 28 or 29 wherein the surfactants are selected from the group consisting of ionic, non-ionic, and zwitterionic surfactants.
31. The method of any of statements 28, 29 or 30 wherein the surfactant is ionic.
32. The method of any of statements 28, 29, 30 or 31 wherein the surfactant is cetyltrimethyl ammonium template or sodium dodecyl sulfate.
33. The method of any of statements 28, 29, or 30 wherein the surfactant is non-ionic.
34. The method of any of statements 28, 29, 30 or 33 wherein the surfactant is selected from the group consisting of polyoxyethylene glycol alkyl ether surfactants, polyethylene glycol surfactants and alkylphenol hydroxypolyethylene surfactants.
35. The method of any of statements 28, 29, or 30 wherein the surfactant is zwitterionic.
36. The method of any of statements 28, 29, 30 or 25 wherein the surfactant is selected from the group consisting of 1,2-dioleoylphosphatidylcholine, 1-palmitoyl-2-oleoyl-sn-glycero-3-phosphocholine, and dipalmitoylphosphatidylcholine.
37. A medical balloon having a drug coating, the drug coating comprising oriented drug crystals wherein the orientation of the drug crystals is not random.
38. The medical balloon of statement 37 wherein the drug crystals are oriented within 45°, more desirably, within 20°, even more desirably, within 10°, even more desirably 5°, and even more desirably, 1° of perpendicular to the surface of the balloon.
39. The medical balloon of any of statements 37 or 38 wherein the balloon comprises a template, the template formed from aggregated surfactants arranged in lamellar structures, the drug crystals disposed in the template.
40. The medical balloon of any of statements 37, 38 or 39 wherein at least 90 percent of the drug crystals are oriented substantially perpendicular to the surface of the balloon.
41. A medical device, at least a portion of which has a drug coating, the drug coating comprising drug crystals on a surface of the medical device, the majority of the drug crystals on the surface of the medical device being oriented drug crystals which extend within 45°, more desirably, within 20°, even more desirably, within 10°, even more desirably 5°, and even more desirably, 1° of an axis which extends at a predetermined, non-zero common angle relative to the surface of the balloon from which the crystals extend.
42. The medical device of statement 41 wherein the oriented drug crystals extend within 15° of an axis which extends perpendicular to the surface of the medical device.
43. The medical device of statement 41 wherein 90 percent or more of the drug crystals on the surface of the medical device are oriented drug crystals which extend within 15° of an axis which extends at a predetermined, non-zero common angle relative to the surface of the device from which the crystals extend.
44. The medical device of statement 43 wherein the oriented drug crystals extend within 15° of an axis which extends perpendicular to the surface of the device.
45. A method of preparing a medical device comprising the steps of:

providing a medical device, the medical device having topological features, each of the features defining a region of the medical device having a depth of at least 5 microns and up to 500 microns;

disposing a drug within the topological features;

forming drug crystals within the topological features;
wherein a majority of the drug crystals are oriented drug crystals which extend within 45°, more desirably, within 20°, even more desirably, within 10°, even more desirably 5°, and even more desirably, 1° of an axis which extends at a predetermined common angle relative to a surface of the medical device.
   46. The method of statement 45 wherein the oriented drug crystals extend within 15° an axis which extends perpendicular to the surface of the medical device.
   47. The method of any of statements 45 or 46 wherein the medical device comprises a polymeric material and the topological features are provided in the polymeric material.
   48. The method of statement 45 or 46 wherein the medical device comprises a template formed from aggregated surfactants arranged in lamellar structures, the topological features provided by the lamellar structures; and removing the template subsequent to forming oriented drug crystals.
   49. The method of statement 48 wherein the template is removed by applying water thereto.
   50. The method of any of statements 48 or 49 wherein the surfactants are selected from the group consisting of ionic, non-ionic, and zwitterionic surfactants.
   51. The method of any of statements 48, 49 or 50 wherein the surfactant is ionic.
   52. The method of any of statements 48, 49, 50 or 51 wherein the surfactant is cetyltrimethyl ammonium template or sodium dodecyl sulfate.
   53. The method of any of statements 48, 49, or 50 wherein the surfactant is non-ionic.
   54. The method of any of statements 48, 49, 50 or 53 wherein the surfactant is selected from the group consisting of polyoxyethylene glycol alkyl ether surfactants, polyethylene glycol surfactants and alkylphenol hydroxypolyethylene surfactants.
   55. The method of any of statements 48, 49, or 50 wherein the surfactant is zwitterionic.
   56. The method of any of statements 48, 49, 50 or 55 wherein the surfactant is selected from the group consisting of 1,2-dioleoylphosphatidylcholine, 1-palmitoyl-2-oleoyl-sn-glycero-3-phosphocholine, and dipalmitoylphosphatidylcholine.
   57. A medical device having a drug coating, the drug coating comprising drug crystals, a majority of the drug crystals being oriented drug crystals wherein the orientation of the drug crystals is not random.
   58. The medical device of statement 57 wherein the oriented drug crystals are oriented within 45°, more desirably, within 20°, even more desirably, within 10°, even more desirably 5°, and even more desirably, 1° of an axis which extends perpendicular to the surface of the medical device.
   59. The medical device of any of statements 57 or 58 comprising a template, the template formed from aggregated surfactants arranged in lamellar structures, the drug crystals disposed in the template.
   60. The medical device of any of statements 57, 58 or 59 wherein at least 90 percent of the drug crystals are oriented within 15° of an axis which extends perpendicular to the surface of the medical device.
   61. The medical device of any of statements 41-44, and 57-60 wherein the medical device is a medical balloon.
   62. The medical device of any of statements 41-44, and 57-60 wherein the medical device is a catheter.
   63. The medical device of any of statements 41-44, and 57-60 wherein the medical device is a stent or a graft.
   64. The medical device of any of statements 41-44, and 57-60 wherein the medical device is an implantable valve.
   65. The method of any of statements 45-56 wherein the medical device is a medical balloon.
   66. The method of any of statements 45-56 wherein the medical device is a catheter.
   67. The method of any of statements 45-56 wherein the medical device is a stent or a graft.
   68. The method of any of statements 45-56 wherein the medical device is an implantable valve.
   69. The catheter of any of statements 1-4 and 17-20, wherein the drug is selected from the group consisting of paclitaxel, olimus drugs and derivates or analogs thereof and combinations thereof.
   70. The catheter of claim 69 wherein the olimus drug is selected from the group consisting of sirolimus, everolimus, zotarolimus, Biolimus A9, deferolimus, AP23572 (Ariad Pharmaceuticals), temsirolimus, tacrolimus, pimecrolimus and derivates or analogs thereof.
   71. The method of any of statements 5-16, 25-36, 45-56 and 65-68 wherein the drug is selected from the group consisting of paclitaxel, olimus drugs and derivates or analogs thereof and combinations thereof.
   72. The method of claim 71 wherein the olimus drug is selected from the group consisting of sirolimus, everolimus, zotarolimus, Biolimus A9, deferolimus, AP23572 (Ariad Pharmaceuticals), temsirolimus, tacrolimus, pimecrolimus and derivates or analogs thereof.
   73. The balloon of any of statements 21-24 and 37-40 wherein the drug is selected from the group consisting of paclitaxel, olimus drugs and derivates or analogs thereof and combinations thereof.
   74. The balloon of claim 73 wherein the olimus drug is selected from the group consisting of sirolimus, everolimus, zotarolimus, Biolimus A9, deferolimus, AP23572 (Ariad Pharmaceuticals), temsirolimus, tacrolimus, pimecrolimus and derivates or analogs thereof.
   75. The medical device of any of statements 41-44, and 57-64 wherein the drug is selected from the group consisting of paclitaxel, olimus drugs and derivates or analogs thereof and combinations thereof.
   76. The medical device of claim 75 wherein the olimus drug is selected from the group consisting of sirolimus, everolimus, zotarolimus, Biolimus A9, deferolimus, AP23572 (Ariad Pharmaceuticals), temsirolimus, tacrolimus, pimecrolimus and derivates or analogs thereof.
   77. A method of preparing a balloon catheter comprising the steps of:

providing a catheter;

providing a balloon material;

providing a microstructure on the balloon material;

wherein either the balloon material is part of a balloon which is disposed about a portion of the catheter; or

subsequent to the step of providing a microstructure on the balloon material, the balloon material is disposed about a portion of the catheter so as to form a balloon.

78. The method of statement 77 wherein the microstructure is provided on the balloon material by laser etching the balloon material.
79. The method of statement 77 wherein the microstructure is printed on the balloon material.

The above examples are for illustrative purposes only, and not limiting to the scope of the present invention. The method steps can be varied as is understood by those of ordinary skill in the art.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

What is claimed is:

1. A catheter comprising a medical balloon having a drug coating, the drug coating comprising drug crystals on a surface of the balloon, a majority of the drug crystals on the surface of the balloon being oriented drug crystals which extend within 45° of a an axis extending at predetermined, non-zero common angle relative to the surface of the balloon from which the crystals extend.

2. The catheter of claim 1 wherein the oriented drug crystals extend within 45° of an axis extending perpendicular to the surface of the balloon.

3. The catheter of claim 1 wherein 90 percent or more of the drug crystals on the surface of the balloon are oriented drug crystals which extend within 45° of a axis extending at a predetermined, non-zero common angle relative to the surface of the balloon from which the crystals extend.

4. The catheter of claim 3 wherein the oriented drug crystals extend within 45° of an axis extending perpendicular to the surface of the balloon.

5. A method of preparing a catheter comprising the steps of:
providing a catheter comprising a medical balloon, the balloon having topological features, the topological features defining one or more regions of the balloon having a depth of at least 50 microns and up to 500 microns;
disposing a drug within the topological features;
forming drug crystals within the topological features;
wherein a majority of the drug crystals are oriented drug crystals which extend within 10° of a predetermined common angle relative to a surface of the balloon.

6. The method of claim 5 wherein the oriented drug crystals extend within 45° of an axis which extends perpendicular to the surface of the balloon.

7. The method of claim 5 wherein the balloon comprises a polymeric material and the topological features are provided in the polymeric material.

8. The method of claim 5 wherein the balloon comprises a template formed from aggregated surfactants arranged in lamellar structures, the topological features provided by the lamellar structures; and removing the template subsequent to forming oriented drug crystals.

9. The method of claim 8 wherein the template is removed by applying water thereto.

10. The method of claim 8 wherein the surfactants are selected from the group consisting of ionic, non-ionic, and zwitterionic surfactants.

11. The method of claim 10 wherein the surfactant is ionic.

12. The method of claim 11 wherein the surfactants are cetyltrimethyl ammonium template or sodium dodecyl sulfate.

13. The method of claim 10 wherein the surfactants are non-ionic.

14. The method of claim 13 wherein the surfactants are selected from the group consisting of polyoxyethylene glycol alkyl ether surfactants, polyethylene glycol surfactants and alkylphenol hydroxypolyethylene surfactants.

15. The method of claim 10 wherein the surfactants are zwitterionic.

16. The method of claim 15 wherein the surfactants are selected from the group consisting of 1,2-dioleoylphosphatidylcholine, 1-palmitoyl-2-oleoyl-sn-glycero-3-phosphocholine, and dipalmitoylphosphatidylcholine.

17. A catheter comprising a medical balloon having a drug coating, the drug coating comprising drug crystals, a majority of the drug crystals being oriented drug crystals wherein the orientation of the drug crystals is not random.

18. The catheter of claim 17 wherein the drug crystals are oriented within 45° of perpendicular to the surface of the balloon.

19. The catheter of claim 17 wherein the balloon comprises a template, the template formed from aggregated surfactants arranged in lamellar structures, the drug crystals disposed in the template.

20. The catheter of claim 17 wherein at least 90 percent of the drug crystals are oriented within 45° of an axis which extends perpendicular to the surface of the balloon.
